(19) United States
(12) Patent Application Publication
(10) Pub. No.: US 2006/0211920 A1
(43) Pub. Date: Sep. 21, 2006

(54) Polymer or plastic load bearing surface for cam actuated retractor clamp

(75) Inventor: Joseph Gordon Bethke, Maple Grove, MN (US)

Correspondence Address: WESTMAN CHAMPLIN & KELLY, P.A., SUITE 1400, 900 SECOND AVENUE SOUTH, MINNEAPOLIS, MN, 55402-3319, US

(73) Assignee: Minnesota Scientific, Inc., St. Paul, MN (US)

(21) Appl. No.: 11/082,358

(22) Filed: Mar. 16, 2005

(51) Int. Cl. A61B 1/32

(52) U.S. Cl. 600/234

(57) ABSTRACT

The present invention includes a surgical clamp having at least one clamping member and a shaft through the clamping member. A camming member engages a load bearing surface on the shaft where the load bearing surface is constructed from a plastic. As the camming member rotates about an axis of rotation, the camming member engages the load bearing surface to position the clamping member into the clamping position.

### BACKGROUND OF THE INVENTION

The present invention relates to a surgical clamp. More particularly, the present invention relates to a surgical clamp with a camming member having a camming surface that engages a load bearing surface constructed from a wear resistant plastic or polymer.

It is known to employ a camming member on a surgical clamp to position the surgical clamp into either a clamping position or non-clamping position. The camming member typically is operably connected to a shaft that is positioned through a clamping member. A handle is typically attached to the camming member to provide leverage in positioning the surgical clamp into the clamping position or the non-clamping position. As the handle is moved, the camming member rotates about an axis of rotation and a camming surface exerts a force upon the shaft which positions the clamping member into the clamping position.

There are numerous designs of camming members that are utilized on surgical clamps including an eccentric surface and an irregularly shaped lobe. However, the camming member and a follower, whether the shaft or a clamping member, are typically constructed from a metallic material such as stainless steel that has a tendency to gall or erode with use over time. Additionally, a significant amount of force is needed to position the camming member into the clamping position because of a high coefficient of friction between the metal camming member and the metal follower, whether the shaft or the clamping member.

Gall resistant metals have been developed to prevent erosion and galling of the camming member and the follower with use over time. However, the specially designed metals typically have high coefficients of friction that require additional force to position the camming member into the clamping position.

### SUMMARY OF THE INVENTION

The present invention includes a surgical clamp having at least one clamping member and a shaft through the clamping member. A camming member engages a load bearing surface on the shaft where the load bearing surface is constructed from a plastic or a polymer. As the camming member rotates about an axis of rotation, the camming member engages the load bearing surface to position the clamping member into the clamping position.

### BRIEF DESCRIPTION OF THE DRAWINGS

FIG. 1 is a perspective view of a surgical clamp of the present invention;

FIG. 2 is an exploded view of the surgical clamp of the present invention;

FIG. 3 is a sectional view of the surgical clamp of the present invention;

FIG. 4 is an alternative embodiment of a surgical clamp of the present invention;

FIG. 5 is a sectional view of an alternative surgical clamp of the present invention;

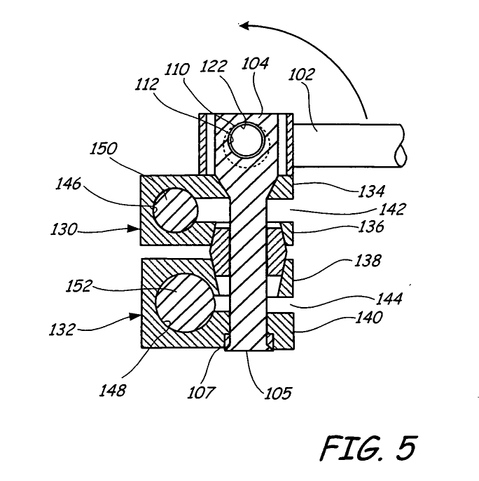

FIG. 6 is another embodiment of a surgical clamp of the present invention; and

FIG. 7 is a sectional view of the alternative embodiment of the surgical clamp.

### DETAILED DESCRIPTION OF THE PREFERRED EMBODIMENTS

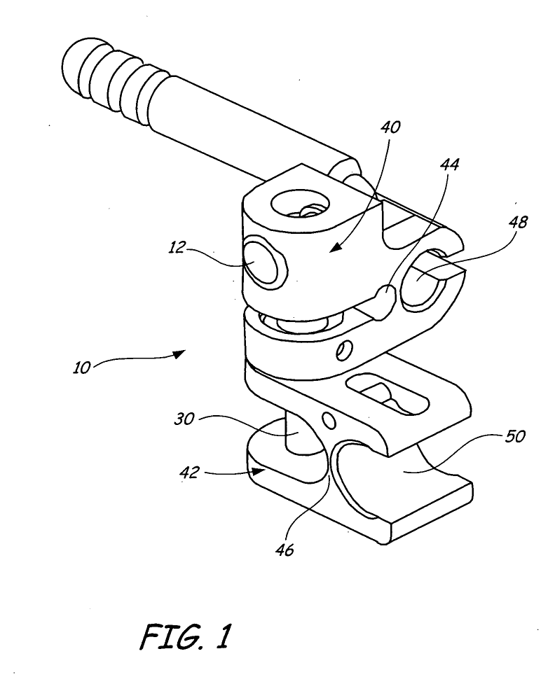

A surgical clamp of the present invention is generally illustrated at 10. The clamp illustrated in FIG. 1 is disclosed in U.S. Pat. No. 5,727,899 which is hereby incorporated by reference in its entirety.

Referring to FIGS. 1-3, the surgical clamp 10 includes a camming member 12 having a camming surface 14 that engages a load bearing surface 20 on an insert 22 that is secured to a shaft 30. The camming surface 14 engages the load bearing surface 20 as the camming member 12 is rotated about an axis of rotation 13 to position the surgical clamp 10 into a clamping position.

The insert 22 is preferably constructed from a polymer or a plastic. The polymer or plastic is wear resistant and heat resistant to allow the clamp 10 to be cycled between the clamping and non-clamping positions without incurring substantial wear while being able to withstand sterilization temperature in an autoclave or similar sterilizing equipment without affecting the properties of the plastic or polymer.

By plastic is meant a polymer material that is typically organic and having a high molecular weight where the material can be shaped by flow. By polymer is meant a substance made by the bonding of simple molecules referred to as monomers into a higher molecular weight molecule. A preferred type of polymer is an engineering polymer. By engineering polymer is meant a polymer that is useful as a material of construction of a part or component of a device.

A non-exhaustive list of engineering polymers that may be utilized in the present invention include fluropolymers such as Teflon® manufactured by E.I. DuPont & Nemours, Inc., polyphenylenesufides such as Ryton® manufactured by Chevron Phillips Chemical Corp., polytetraflouroethylene such as Rulon® manufactured by Saint-Gobain Performance Plastics Corporation and polyetheretherketones such as PEEK® manufactured by Vitrix PLC. The preferred engineering polymer for constructing the insert 22 with the load bearing surface 20 for engagement with the camming surface 14 is a polyetheretherketone.

Polyetheretherketone is suitable material of construction of the insert 22 with load bearing surface 20 because polyetheretherketone is heat and wear resistant. An additional property that makes polyetheretherketone a suitable material of construction for the load bearing surface 20 is a low coefficient of friction.

Most Materials, such as stainless steel, either are wear resistant and have a high coefficient of friction. Alternatively, the materials tend to have a low coefficient of friction but have a tendency to wear with use. Polyetheretherketone has the unique combination of wear resistance and a low coefficient of friction.

Surgical clamps such as the clamp 10 are manipulated from the clamping position to the non-clamping position and back into the clamping position thousands of times over the life of the clamp and require that the load bearing surface 20 as well as the camming surface 14 to be wear resistant for the clamp to properly function over time. Further, the material of construction of the clamp must be able to withstand autoclave temperatures so that the clamp can be sterilized.

An engineering polymer such as polyetheretherketone is well suited as the material of construction for the load bearing surface 20 for engaging the camming surface 14 because the polyetheretherketone is heat and wear resistant. The insert 22 having the load bearing surface 20 constructed of polyetheretherketone also has a coefficient of friction that allows the camming member 12 to be rotated into the clamping position with about seventy percent less force when compared to a stainless steel camming surface engaging a stainless steel load bearing surface. However, other polymer materials of construction are within the scope of the present invention.

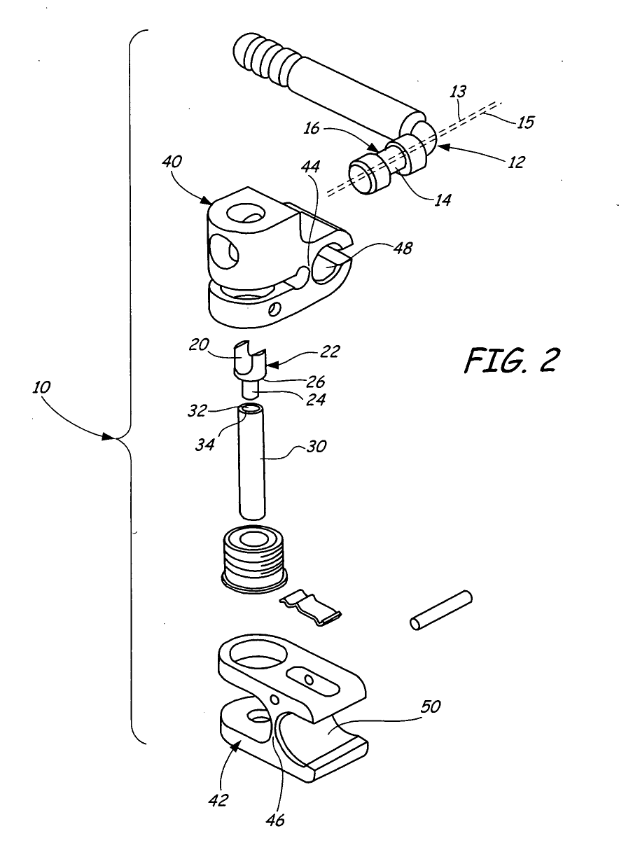

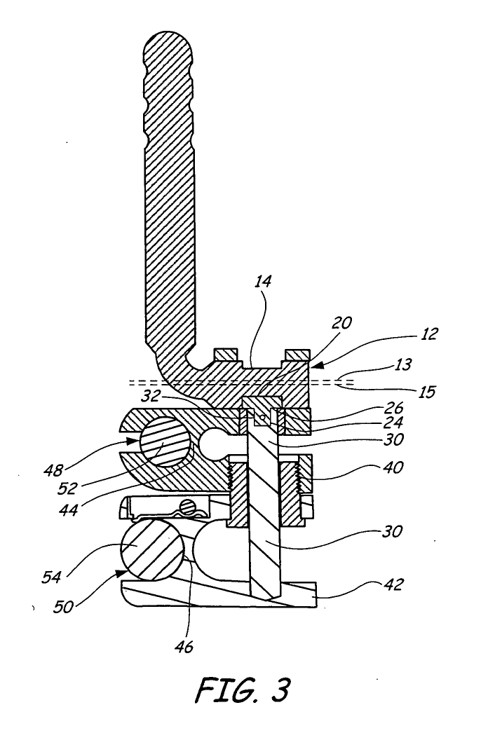

Referring to FIGS. 2 and 3, the insert 22 extends from an end 34 of the shaft 30 and includes the concave load bearing surface 20 that engages the camming surface 14 of the camming member 12. The insert 22 includes a cylindrical peg 24 that is positioned into a cavity 32 in the end 34 of the shaft 30 where the cylindrical peg 24 is movable within the cavity 32. A shoulder 26 engages the end 34 to support the insert 22 on the shaft 30.

The insert 22 may also be fixedly attached to the shaft 30. The cylindrical peg 24 may be glued or cemented within the cavity 34, as well as frictionally retained within the cavity 34 with a compression fit to fixedly attach the insert 22 to the shaft 30.

The camming surface 14 is defined by an eccentric cylinder 16 having an axis 15 that is offset from the axis of rotation 13 of the camming member 12. The camming surface 14 engages the concave load bearing surface 20 as the camming member 12 is rotated from the non-clamping position to the clamping position and forces the load bearing surface 20 from the axis of rotation 13 of the camming member 12. As the load bearing surface 20 is forced from the axis of rotation 13, the shaft 20 applies a force to upper and lower clamping members 40, 42 which causes fulcrum portions 44, 46 on the upper and lower clamping members 40, 42 to flex. The fulcrum portions 44, 46 flex and constrict clamping sockets 48, 50 in the upper and lower clamping members 40, 42 to retain elongated members 52, 54 therein.

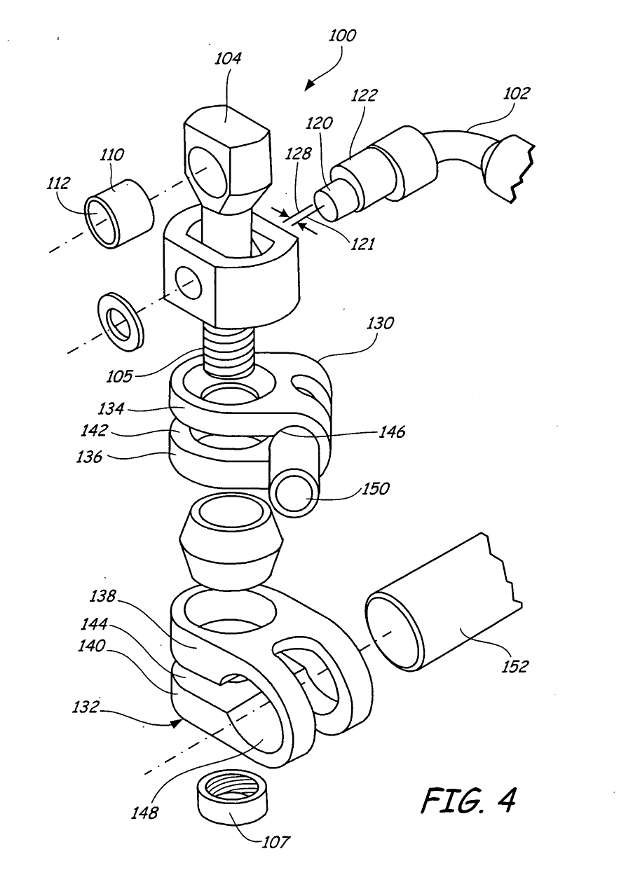

Referring to FIG. 4, an alternatively designed clamp 100 includes a camming member 102 and a shaft 104 with a through bore 106. The clamp illustrated in FIG. 4 is disclosed in U.S. Pat. No. 5,792,046 which is hereby incorporated by reference in its entirety.

A liner 110 constructed of the wear and heat resistant polymer is positioned within the through bore 106 in the shaft 104. A distal portion 120 of the camming member 102 is positioned through an aperture 112 in the liner 110 such that a camming surface 122 engages the liner 110. The liner 110 constructed from the engineering polymer provides a wear and heat resistant load bearing surface while reducing the amount of force required to position the clamp 100 into the clamping position from the non-clamping position.

The camming surface 122 has an axis 123 that is offset from the axis of rotation 121 of the camming member 102. As the camming member 102 is rotated, the camming surface 122 engages the liner 110 and draws a distal end 105 of the shaft 104 toward the camming member 102. As the distal end 105 of the shaft 104 is drawn toward the camming member 104, a nut 107 threadably attached to the distal end 105 of the shaft applies a force to the lower clamping member 132 and an upper clamping member 130.

The upper clamping member 130 and the lower clamping member 132 are positioned into clamping positions by forcing upper and lower legs 134, 136, 138, 140 separated by slots 142, 144 toward each other, all respectively. As the upper and lower legs 134, 136, 138, 140 are forced toward each other, clamping bores 146, 148 are constricted and secure support rods 150, 152 therein.

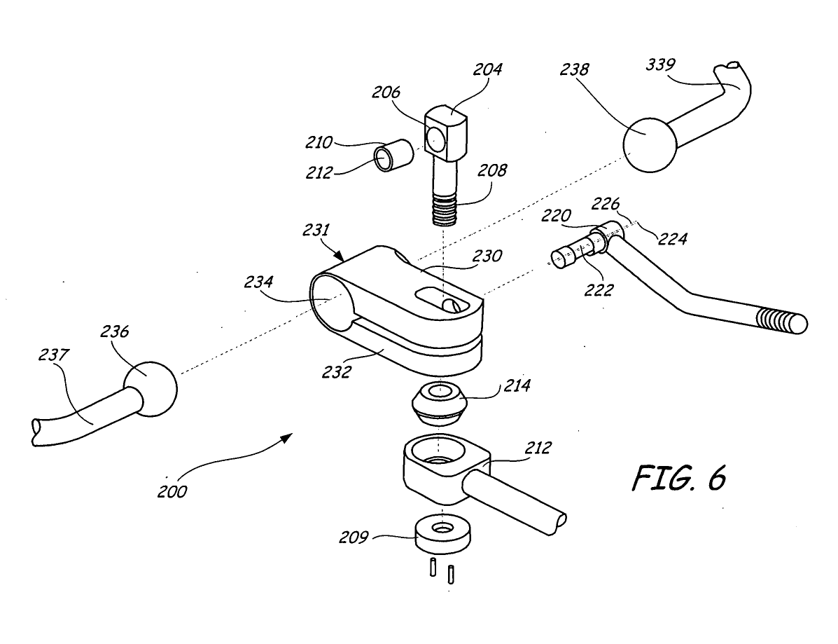

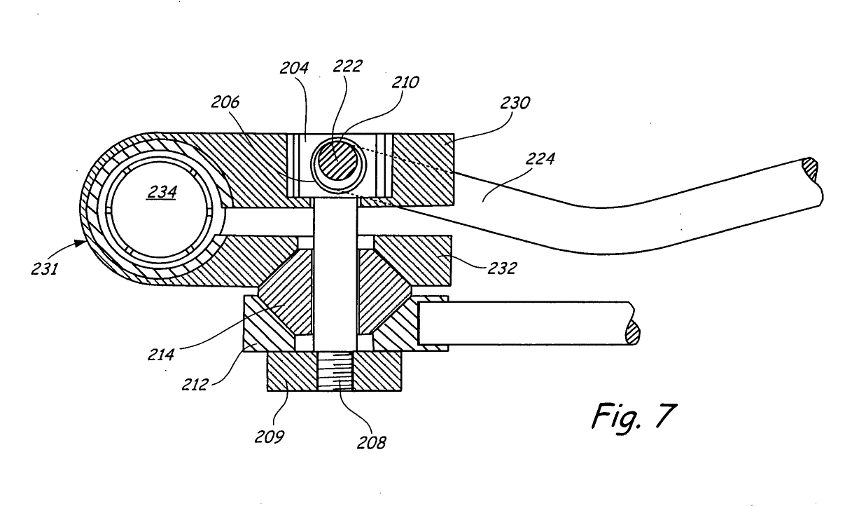

Referring to FIGS. 6 and 7, a liner 210 constructed of the heat resistant and wear resistant polymer can be employed in a clamp 200. The clamp 200 engages and secured support arms 237, 239 with pivot balls 236, 238 in selected positions. The clamp 200 illustrated in FIG. 6 is disclosed in U.S. Pat. Nos. 5,899,627 and 6,264,396 both of which are incorporated by reference in their entireties.

The liner 210 is positioned within a through bore 206 in a shaft 204. The liner 210, constructed of the wear and heat resistant polymer having a low coefficient of friction, allows the camming member 220 to be rotated into the clamping position with significantly less force than a clamp without the liner 210.

The shaft 204 is positioned through bores in a clamping member 231. With the shaft positioned within the clamping member 231, a camming member 220 with a camming surface 222 is positioned through a through bore 212 in the line 210 such that the camming surface 222 engages the liner 210. A threaded end 208 of the shaft 204 engages a nut 209 that retains a second clamping member 212 and the clamping member 231 that are separated by a spacer 214.

The clamp 200 is placed into the clamping position by rotating a camming member 220 having the eccentric camming surface 222 with an axis 226 that is offset from the axis 224 of rotation of the camming member 220. As the camming member 220 is rotated, the eccentric surface 222 engages the liner 210 and draws the shaft 204 toward the axis of rotation 224 of the camming member 220 and positions the clamp 200 into the clamping position.

Although the present invention has been described with reference to preferred embodiments, workers skilled in the art will recognize that changes may be made in form and detail without departing from the spirit and scope of the invention.

What is claimed is:

1. A surgical clamp comprising:
at least one clamping member;
a shaft through the clamping member and having a load bearing surface constructed from a plastic; and
a camming member engaging the load bearing surface such that the camming member rotates about an axis of rotation and engages the plastic load bearing surface to position the clamping member into a clamping position.

2. The surgical clamp of claim 1 and wherein the plastic comprises an engineering polymer.

3. The surgical clamp of claim 2 and wherein the engineering polymer comprises a fluropolymer, a polyphenylenesufide, a polytetraflouroethylene or a polyetheretherketone.

4. The surgical clamp of claim 2 and wherein the engineering polymer comprises polyetheretherketone.

5. The surgical clamp of claim 1 and further comprising:
a first clamping member; and
a second clamping member and wherein the shaft being positioned through the first and second clamping members such that as the camming member engages the load bearing surface the first and second clamping members are positioned into clamping positions.

6. The surgical clamp of claim 1 and wherein the clamping member includes a clamping bore.

7. The surgical clamp of claim 1 and wherein the clamping member includes a clamping socket.

8. The surgical clamp of claim 1 and wherein the camming member draws the shaft toward the axis of rotation to position the clamping member into the clamping position.

9. The surgical clamp of claim 1 and wherein the camming member forces the shaft away from the axis of rotation to position the clamping member into the clamping position.

10. A surgical clamp comprising:
at least one clamping member;
a shaft through the clamping member;
an insert constructed of a polymer and being attached to the shaft and wherein the insert includes a load bearing surface; and
a camming member having a camming surface and an axis of rotation and wherein the camming surface engages the load bearing surface on the insert and wherein the polymer has a coefficient of friction that is less than a coefficient of friction of a material of construction of the shaft such that less force is required to rotate the camming member about the axis of rotation and into a clamping position.

11. The surgical clamp of claim 10 and wherein the insert attaches to an end of the shaft and wherein the load bearing surface comprises a concave configuration.

12. The surgical clamp of claim 10 and wherein the insert comprises a sleeve positioned within a through bore in the shaft and wherein the camming surface engages the sleeve.

13. The surgical clamp of claim 10 and wherein the polymer comprises an engineering polymer.

14. The surgical clamp of claim 13 and wherein the engineering polymer comprises a fluropolymer, a polyphenylenesufide, a polytetraflouroethylene or a polyetheretherketone.

15. The surgical clamp of claim 13 and wherein the engineering polymer comprises a polyetheretherketone.

16. An improved surgical clamp having at least one clamping member, a shaft through the clamping member and a camming member having a camming surface engaging the shaft and wherein the camming member rotates to position the clamp into the clamping position, the improvement comprising:
an insert constructed from a polymer and being attached to the shaft wherein the insert comprises a load bearing and wherein the camming surface engages the load bearing surface to reduce the amount of force required to position the clamp into the clamping position.

17. The surgical clamp of claim 16 and wherein the polymer comprises an engineering polymer.

18. The surgical clamp of claim 17 and wherein the engineering polymer comprises a fluropolymer, a polyphenylenesufide, a polytetraflouroethylene or a polyetheretherketone.

19. The surgical clamp of claim 17 and wherein the engineering polymer comprises a polyetheretherketone.

20. The surgical clamp of claim 16 and wherein the insert attaches to an end of the shaft comprises an end comprising a concave load bearing surface for engaging the camming surface.

21. The surgical clamp of claim 16 and wherein the insert comprises a sleeve positioned within a through bore in the shaft and wherein the camming surface engages the sleeve.
